(19) United States
(12) Patent Application Publication
(10) Pub. No.: US 2025/0328148 A1
(43) Pub. Date: Oct. 23, 2025

(54) MANAGEMENT SYSTEM, MANAGEMENT METHOD, AND STORAGE MEDIUM

(71) Applicant: TOYOTA JIDOSHA KABUSHIKI KAISHA, Toyota-shi (JP)

(72) Inventors: Satoshi TOYOSHIMA, Toyota-shi (JP); Shiro ODA, Anjo-shi (JP); Takeshi MATSUI, Nisshin-shi (JP)

(73) Assignee: TOYOTA JIDOSHA KABUSHIKI KAISHA, Toyota-shi (JP)

(21) Appl. No.: 19/062,175

(22) Filed: Feb. 25, 2025

(30) Foreign Application Priority Data
Apr. 22, 2024 (JP) 2024-068922

(51) Int. Cl. G05D 1/646; G05D 109/10

(52) U.S. Cl. CPC G05D 1/646; G05D 2109/10

(57) ABSTRACT

The management system according to the present embodiment is a management system that manages an autonomous mobile robot and a plurality of accessory units that enable a mobile robot to execute a plurality of different services by being used in combination with the mobile robot, and acquires service information related to a service executed by the mobile robot, acquires a plurality of control parameters set for each service based on the service information, and controls an operation of the mobile robot according to the plurality of control parameters. The management system may use a machine learning model such as deep learning.

### CROSS-REFERENCE TO RELATED APPLICATION

This application claims priority to Japanese Patent Application No. 2024-068922 filed on Apr. 22, 2024, incorporated herein by reference in its entirety.

### BACKGROUND

### 1. Technical Field

The present disclosure relates to a management system, a management method, and a storage medium.

### 2. Description of Related Art

WO 2023/86665 discloses an autonomous mobile robot that tows a wheeled cart. The autonomous mobile robot includes a traction arm that tows the wheeled cart. The autonomous mobile robot tows the wheeled cart with the traction arm inserted under the wheeled cart.

### SUMMARY

In WO 2023/86665, the autonomous mobile robot can execute a transport service to transport the wheeled cart. However, there is a desire to allow such a mobile robot to execute services other than the transport service.

An aspect of the present embodiment provides
a management system that manages an autonomous mobile robot and a plurality of accessory units to be used in combination with the autonomous mobile robot to enable the autonomous mobile robot to execute a plurality of different services, the management system being configured to:
acquire service information related to a service executed by the autonomous mobile robot;
acquire a plurality of control parameters set for each service based on the service information; and
control operation of the autonomous mobile robot according to the control parameters.

Another aspect of the present embodiment provides
a management method of managing an autonomous mobile robot and a plurality of accessory units to be used in combination with the autonomous mobile robot to enable the autonomous mobile robot to execute a plurality of different services, the management method including:
acquiring service information related to a service executed by the autonomous mobile robot;
acquiring a plurality of control parameters set for each service based on the service information; and
controlling operation of the autonomous mobile robot according to the control parameters.

Still another aspect of the present embodiment provides
a medium storing a program that causes a computer to execute a management method of managing an autonomous mobile robot and a plurality of accessory units to be used in combination with the autonomous mobile robot to enable the autonomous mobile robot to execute a plurality of different services, in which
the management method includes:
acquiring service information related to a service executed by the autonomous mobile robot;
acquiring a plurality of control parameters set for each service based on the service information; and
controlling operation of the autonomous mobile robot according to the control parameters.

According to the present disclosure, it is possible to provide a management system, a management method, and a medium storing a program that can perform appropriate control according to a service when an autonomous mobile robot is used together with an accessory unit.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic diagram illustrating an overall configuration of a management system according to the present embodiment;

FIG. 2 is a block-diagram illustrating a control system of the management system; and

FIG. 3 is a flowchart illustrating a management method.

### DETAILED DESCRIPTION OF EMBODIMENTS

Hereinafter, the present disclosure will be described through embodiments of the disclosure. However, the disclosure according to the claims is not limited to the following embodiments. Moreover, all of the configurations described in the embodiments are not necessarily indispensable as means for solving the issue.

### Overall Configuration

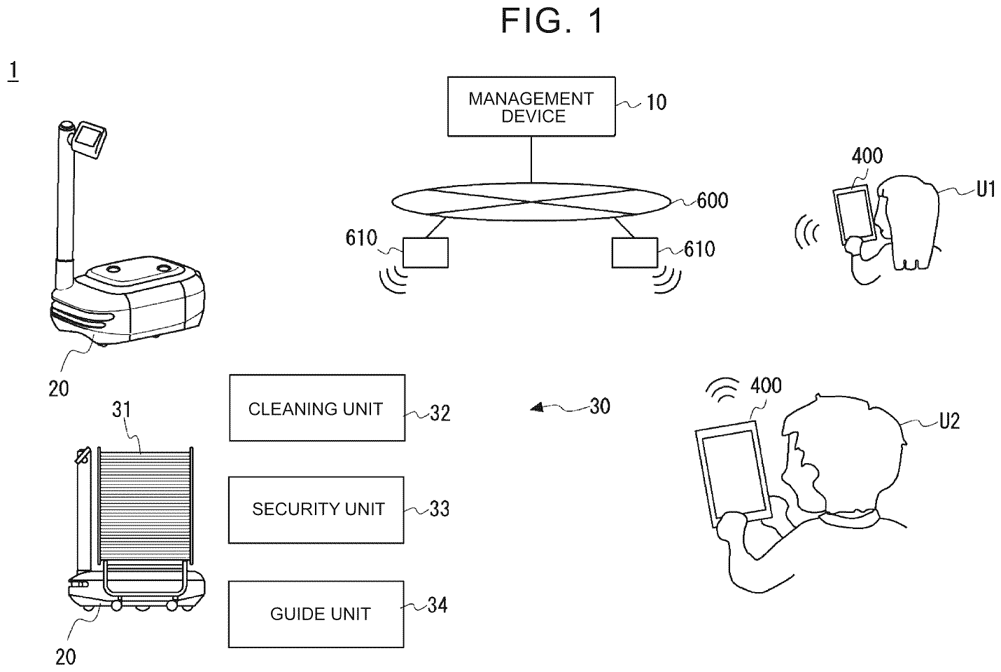

The management system according to the present embodiment is a system for managing an autonomous mobile robot capable of executing a plurality of services (also referred to as tasks). FIG. 1 is a schematic diagram illustrating a configuration of a management system 1. The management system 1 includes a mobile robot 20, a host management device 10, an accessory unit 30, a network 600, a communication unit 610, and a user terminal 400. The management system 1 is a system for managing one or a plurality of mobile robots 20 and an accessory unit 30 thereof.

The accessory unit 30 is used in combination with the mobile robot 20 to execute each service. For example, the accessory unit 30 is a transport unit 31, a cleaning unit 32, a security unit 33, and a guide unit 34. The accessory unit 30 is not limited thereto, and may include a unit for other services. The accessory unit 30 may be devoid of one or more of the above four.

The mobile robot 20 is an autonomous mobile robot and executes a plurality of services such as transportation, cleaning, security, and guidance. The mobile robot 20 autonomously moves medical welfare facilities such as hospitals, rehabilitation centers, nursing facilities, and residential facilities for the elderly. Moreover, the system according to the present embodiment can also be used in commercial facilities such as shopping malls. The mobile robot includes wheels, a chassis, a motor, a sensor, a battery, a controller, and the like.

The mobile robot 20 mounts any one of the transport unit 31, the cleaning unit 32, the security unit 33, and the guide unit 34 in accordance with a service to be executed. For example, the mobile robot 20 mounts the transport unit 31 when the transport service is executed. When executing a cleaning service, a security service, or a guidance service, the mobile robot 20 is equipped with a cleaning unit 32, a security unit 33, or a guide unit 34, respectively. As described above, the accessory unit 30 is prepared in advance for each service to be executed.

The mobile robot 20 selectively attaches any one of the transport unit 31, the cleaning unit 32, the security unit 33, and the guide unit 34. The mobile robot 20 selectively uses the accessory unit 30 according to the service to be executed. The mobile robot 20 may be capable of mounting two or more accessory units 30 at the same time. Further, the mobile robot 20 may be configured to be able to attach the accessory unit 30 only by its own operation, but may be configured to be assisted by a user or the like.

The transport unit 31 is an accessory unit 30 for transporting a conveyed object. For example, the transport unit 31 is a wagon or a cart with wheels, and is capable of mounting a conveyed object therein. The carriage portion of the mobile robot 20 serves as a stage on which a wagon or the like is mounted. The mobile robot 20 has an elevating function for lifting the transport unit 31. When the carriage portion of the mobile robot 20 enters the lower side of the transport unit 31, an elevating stage or the like lifts the transport unit 31. As a result, the mobile robot 20 mounts the transport unit 31. The mobile robot 20 can convey the conveyed object stored in the transport unit 31 to the destination. The mobile robot 20 can execute the conveyance service by mounting the transport unit 31. The transport unit 31 is used for serving a table in a restaurant, a medical welfare facility, or the like, and for a lower table. The transport unit may be used for transporting parts in a factory, transporting samples in a hospital, transporting linen in a hotel, and the like.

The cleaning unit 32 is an accessory unit 30 for cleaning a facility. The cleaning unit 32 includes a cleaner that sucks dust and the like. Alternatively, the cleaning unit 32 includes a brush, a pad for wiping the floor, a mop, and the like. When the mobile robot 20 moves with the cleaning unit 32 mounted thereon, the floor surface is cleaned. That is, in the region where the mobile robot 20 has moved, the floor surface is cleaned. The mobile robot 20 can execute the cleaning service by mounting the cleaning unit 32. The cleaning unit 32 may also be a remotely controllable robotic cleaner. In this case, the mobile robot 20 performs the cleaning service by remotely controlling the cleaning unit 32 by wireless communication.

The security unit 33 is an accessory unit 30 for securing the facility. The security unit 33 includes, for example, various sensors for detecting intruders and abnormalities. For example, the security unit 33 includes a camera, an infrared camera, a ranging sensor, an optical sensor, a thermal sensor, and a smoke sensor as sensors. A lighting device for illuminating an intruder or an abnormal portion may be provided. The accessory unit 30 may have an alarm function or the like for notifying when an abnormality is detected. The mobile robot 20 can execute the security service by wearing the security unit 33.

The guide unit 34 is an accessory unit 30 for guiding the facility. For example, the guide unit 34 guides the visitor of the facility a route to the destination or the like. The guide unit 34 has an input device for a visitor to input a destination. The input device includes a touch panel and buttons. The input device also includes a microphone for voice input. The mobile robot 20 moves to a destination in order to guide a visitor. The guide unit 34 may include a display device for displaying a route and a speaker for outputting a voice of the route.

As described above, the accessory unit 30 is used in combination with the mobile robot 20, so that the mobile robot 20 can execute a plurality of different services. That is, the mobile robot 20 executes a service corresponding to the accessory unit 30.

A plurality of accessory units 30 for one service may be provided. In the management system 1, for example, two or more transport units 31 may be provided. In this case, the two mobile robots 20 can simultaneously execute the transport service using the transport unit 31. Further, a plurality of types of accessory units 30 may be prepared for one service. For example, the shape and size of the transport unit 31 may be different depending on the contents of the conveyed material.

The user U1 or the user U2 can use the user terminal 400 to make a service request such as a conveyance request for a conveyed object. For example, the user terminal 400 is a tablet computer, smart phone, or the like. The user terminal 400 only needs to be an information processing device capable of wireless or wired communication.

In the present embodiment, the mobile robot 20 and the user terminals 400 are connected to the host management device 10 via the network 600. The mobile robot 20 and the user terminals 400 are connected to the network 600 via the communication units 610. The network 600 is a wired or wireless local area network (LAN) or wide area network (WAN). The host management device 10 is connected to the network 600 by wire or wirelessly. The communication unit 610 is, for example, a wireless LAN unit installed in each environment. The communication unit 610 may be, for example, a general purpose communication device such as a Wi-Fi router.

Various signals transmitted from the user terminals 400 of the users U1 and U2 are once sent to the host management device 10 via the network 600, and transmitted from the host management device 10 to the target mobile robot 20. Similarly, various signals transmitted from the mobile robot 20 are once sent to the host management device 10 via the network 600, and transmitted from the host management device 10 to the target user terminal 400. The host management device 10 is a server connected to each equipment, and collects data from each equipment. The host management device 10 is not limited to a physically single device, and may have a plurality of devices that performs distributed processing. Further, the host management device 10 may be distributedly provided in an edge device such as the mobile robot 20. For example, a part or all of the management system 1 may be mounted on the mobile robot 20.

The user terminal 400 and the mobile robot 20 may transmit and receive signals without the host management device 10. For example, the user terminal 400 and the mobile robot 20 may directly transmit and receive signals by wireless communication. Alternatively, the user terminal 400 and the mobile robot 20 may transmit and receive signals via the communication unit 610.

It is assumed that a plurality of mobile robots 20 are used in a facility. The host management device 10 assigns a service to each mobile robot 20. Each of the mobile robots 20 is equipped with an accessory unit 30 corresponding to the assigned service, and executes the service. The service executed by the mobile robot 20 may be entered by a user U1 or a user U2, or may be scheduled in advance. For example, a user U1 or the like operates the user terminal 400 to make a service request. A user U1 or the like can enter the type of service to be executed. A user U1 or the like may enter an area, a time-zone, or the like in which the service is executed.

In such an overall configuration, each element of the management system 1 can be distributed among the mobile robot 20, the user terminal 400, and the host management device 10 to construct the management system 1 as a whole. Further, it is possible to collect substantial elements for achieving the transportation of the transported object in a single device to construct the system. The host management device 10 controls one or more mobile robots 20.

### Control System

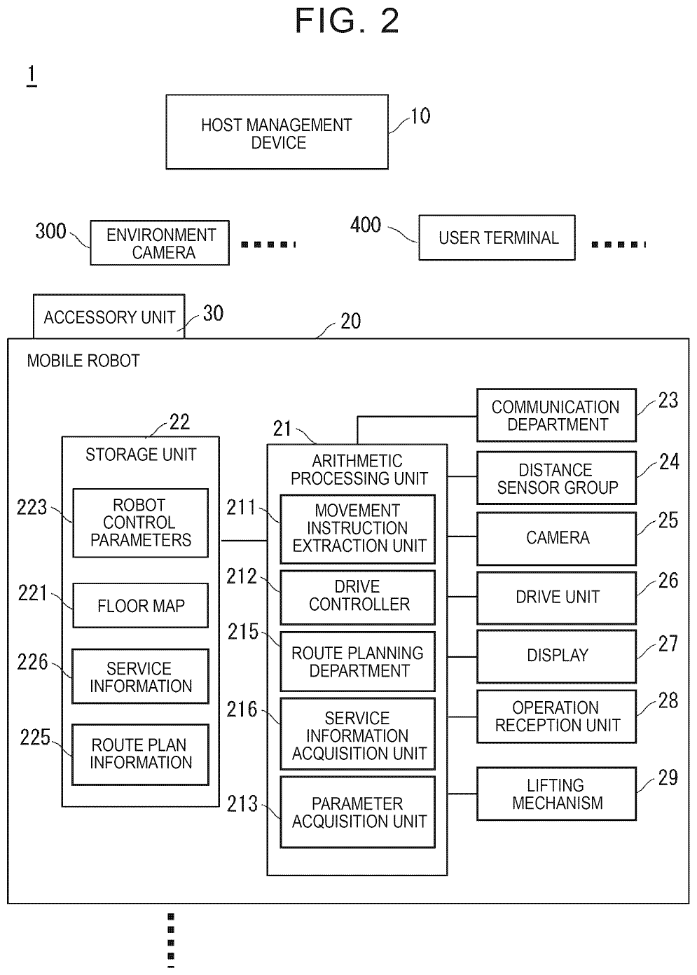

FIG. 2 is a block diagram illustrating a control system of the management system 1 according to the present embodiment. As illustrated in FIG. 2, the management system 1 includes a host management device 10, a mobile robot 20, an accessory unit 30, an environment camera 300, and a user terminal 400. In FIG. 2, the network 600 and the communication unit 610 are omitted. Although only one mobile robot 20 is illustrated in FIG. 2, a plurality of mobile robots 20 may be used.

The host management device 10 includes a personal computer or the like, and performs an operation for controlling and managing the mobile robot 20. The host management device 10 can be implemented as, for example, a device capable of executing a program such as a central processing unit (CPU: Central Processing Unit) of a computer. Various functions can also be realized by the program. For example, the host management device 10 manages the mobile robot 20 so as to operate efficiently. For example, upon receiving a service request from the user terminal 400 or the like, the host management device 10 selects one mobile robot 20 from the plurality of mobile robots 20 and instructs the mobile robot 20 to execute the service. Alternatively, the host management device 10 instructs the mobile robot 20 to use the accessory unit 30.

A plurality of environment cameras 300 are installed in a facility where the mobile robot 20 travels. For example, the environment cameras 300 are each installed in a passage, a hallway, an elevator, an entrance, etc. in the facility.

The environment cameras 300 acquire images of ranges in which the mobile robot 20 moves. In the management system 1, the image acquired by the environment camera 300 and information based on the image are collected by the host management device 10. Alternatively, an image or the like acquired by the environment camera 300 may be directly transmitted to the mobile robot 20. The environment cameras 300 may be surveillance cameras or the like provided in a passage or an entrance/exit in the facility.

In the management system 1, the mobile robot 20 performs route planning based on the service information. Based on the route plan information created by the mobile robot 20, the mobile robot autonomously moves toward the destination. The mobile robot 20 autonomously moves toward the destination using sensors, floor maps, position information, and the like provided in the mobile robot 20 itself. Of course, a part of the processing of the mobile robot 20 may be performed by the host management device 10.

For example, the mobile robot 20 travels so as not to come into contact with surrounding equipment, objects, walls, and people (hereinafter collectively referred to as peripheral objects). Specifically, the mobile robot 20 detects a distance to a surrounding object and travels in a state of being separated from the surrounding object by a certain distance (also referred to as a distance threshold or a margin distance) or more. When the distance from the peripheral object becomes equal to or less than the distance threshold value, the mobile robot 20 decelerates or stops. With this configuration, the mobile robot 20 can travel without coming into contact with the peripheral objects. Since contact can be avoided, safe and efficient transportation is possible.

The mobile robot 20 includes an arithmetic processing unit 21, a storage unit 22, a communication unit 23, a distance sensor group 24, a camera 25, a drive unit 26, a display unit 27, and an operation reception unit 28. Although FIG. 2 shows only typical processing blocks provided in the mobile robot 20, the mobile robot 20 also includes many other processing blocks that are not shown.

The communication unit 23 is a communication interface for communicating with the host management device 10, the environment camera 300, or another mobile robot 20. The communication unit 23 communicates with the host management device 10 or the like using, for example, a wireless signal. The distance sensor group 24 is, for example, a proximity sensor, and outputs proximity object distance information indicating a distance from an object or a person that is present around the mobile robot 20. The camera 25, for example, captures an image for grasping the surrounding situation of the mobile robot 20. The mobile robot 20 may identify a surrounding object based on an image of the camera 25 or the environment camera 300.

The drive unit 26 includes a motor that drives drive wheels provided in the mobile robot 20. Note that, the drive unit 26 may include an encoder or the like that detects the number of rotations of the drive wheels and the drive motor thereof. The position of the mobile robot 20 (current position) may be estimated based on the output of the above encoder. The mobile robot 20 detects its current position and transmits the information to the host management device 10.

The display unit 27 and the operation reception unit 28 are realized by a touch panel display. The display unit 27 displays a user interface screen that serves as the operation reception unit 28. Further, the display unit 27 may display information indicating the destination of the mobile robot 20 and the state of the mobile robot 20. The operation reception unit 28 receives an operation from the user. The operation reception unit 28 includes various switches provided on the mobile robot 20 in addition to the user interface screen displayed on the display unit 27.

The lifting mechanism 29 lifts and lowers a carriage unit for mounting the transport unit 31. The lifting mechanism 29 includes a motor and a lifting stage that is moved up and down by the motor. The elevating stage is provided in a carriage portion of the mobile robot 20. In a state in which the stage is directly below the transport unit 31, the lifting mechanism 29 raises the stage, and thus the transport unit 31 is raised. Since the wheels of the transport unit 31 are separated from each other, the transport unit 31 is stacked on the mobile robot 20 (see FIG. 1). When the mobile robot 20 transports the conveyed object to the destination, the lifting mechanism 29 lowers the stage. As a result, the transport unit 31 is lowered from the stage.

The storage unit 22 stores a floor map 221, robot control parameters 223, and service information 226. The information illustrated in FIG. 2 is a part of the information stored in the storage unit 22, and may include information other than the floor map 221, the robot control parameter 223, and the service information 226 illustrated in FIG. 2.

The floor map 221 is map information of a facility in which the mobile robot 20 moves. The floor map 221 may download the floor map 221 from the host management device 10, for example. The floor map 221 may be created in advance. Further, the floor map 221 may not be the map information of the entire facility but may be the map information including part of the area in which the mobile robot 20 is scheduled to move.

The route plan information 225 includes route plan information planned by the route planning unit 215. The route plan information 225 includes, for example, information indicating a destination and route information to the destination. The route plan information 225 may include information such as a departure point, a scheduled start time of a service, and a scheduled end time. The route plan information may include information on a passing point or a passing place in the moving route. In the route plan information 225, the above-described various types of information may be associated with each service. The route plan information 225 may include at least a part of the service information or the like inputted from the user U1.

The service information 226 includes information on a service executed by the mobile robot 20. The service information 226 includes information on a type of service, a start time, an end time, a time zone to be executed, an area to be executed, and the like. Further, the service information 226 may include information indicating the execution order, whether the execution has been completed, or information regarding the accessory unit 30.

The robot control parameter 223 is a parameter used for controlling the operation of the mobile robot 20. The robot control parameters 223 include a threshold distance or the like between the mobile robot 20 and the surrounding object. The threshold distance is a margin distance (also referred to as a virtual bumper distance) for avoiding contact with a surrounding object including a person. Further, the robot control parameter 223 may include speed information related to a speed such as a speed upper limit value of the mobile robot 20. The robot control parameters 223 may include at least one of speed information, height of the lifting mechanism, virtual bumper distance, suspension hardness, turning radius, allowable inclination angle, travelable environment designation, battery consumption mode, and wireless sensitivity.

The arithmetic processing unit 21 performs arithmetic used for controlling the mobile robot 20. The arithmetic processing unit 21 can be implemented as a device capable of executing a program such as a central processing unit (CPU) of a computer, for example. Various functions can also be realized by the program. The arithmetic processing unit 21 includes a movement command extraction unit 211, a drive control unit 212, a parameter acquisition unit 213, a route planning unit 215, and a service information acquisition unit 216. Although FIG. 2 shows only typical processing blocks included in the arithmetic processing unit 21, the arithmetic processing unit 21 includes processing blocks that are not shown.

The movement command extraction unit 211 extracts a movement command from the control signal and the route plan information 225 given from the host management device 10. For example, the movement command includes information on the next passing point. For example, the control signal may include information on the coordinates of the passing points and the passing order of the passing points. The movement command extraction unit 211 extracts these types of information as a movement command.

The drive control unit 212 controls the drive unit 26 such that the drive unit 26 moves the mobile robot 20 based on the movement command given from the movement command extraction unit 211. For example, the drive unit 26 includes drive wheels that rotate in accordance with a control command value from the drive control unit 212. The movement command extraction unit 211 extracts a movement command so that the mobile robot 20 moves along the movement path indicated by the route plan information 225. The drive unit 26 rotationally drives the drive wheels. The mobile robot 20 autonomously moves toward a destination or a waypoint. The mobile robot 20 may estimate its own position and transmit a signal indicating that it has passed through a waypoint or the like to the host management device 10. Thus, the host management device 10 can manage the current position and the transportation status of each mobile robot 20.

The route planning unit 215 performs route planning of the mobile robot 20. Upon receiving the service request from the user terminal 400 or the host management device 10, the route planning unit 215 performs a route plan for executing the service based on the service information. For example, the route planning unit 215 searches for a route to the area where the service is to be performed or a route in the service. As a result, a movement path in the floor map 221 is obtained. The route plan calculated by the route planning unit 215 is written in the storage unit 22 as route plan information 225.

Specifically, the route planning unit 215 refers to the floor map 221, the service information 226, and the like that are already stored in the storage unit 22, and sets a departure point, a passing point, and a destination. The departure point is a current position or the like. The transit place is the detachment position of the accessory unit 30, etc. The destination is, for example, a place where the service is executed. Further, in a case where an area for executing a cleaning service, a monitoring service, or the like is set, the route planning unit 215 performs route planning using a point in the area where the service is performed as a via point or a destination.

The service information acquisition unit 216 acquires service information from the host management device 10 or the user terminal 400. The service information acquisition unit 216 acquires service information related to a service assigned to the mobile robot 20. For example, when a service requested by the user U1 and a time/area thereof are inputted, the service information acquisition unit 216 acquires the content as service information. The service information acquisition unit 216 may acquire the service information from the host management device 10, or may directly acquire the service information from the user terminal 400. The service information acquisition unit 216 may acquire the service information 226 from a preset schedule. In addition, the service information acquisition unit 216 may acquire the service information from an image of the camera 25 or the environment camera 300.

Further, when the user U1 or the like attaches the accessory unit 30 to the mobile robot 20, the sensor provided in the mobile robot 20 detects the accessory unit 30, so that the service to be executed can be specified. A RFID tag, a QR code (registered trademark), or the like for specifying a service/accessory unit 30 may be mounted on the accessory unit 30. In addition, the accessory unit 30 may transmit a signal for specifying a service to the mobile robot 20.

In a case where the scheduled execution of the service (such as the scheduled start time and the scheduled end time) is scheduled in advance, the service information acquisition unit 216 may specify the service from the schedule information. The service information acquisition unit 216 writes the acquired information on the service into the storage unit 22 as the service information 226. In addition, when two or more services are allocated in succession, the service information may include an execution order thereof.

The parameter acquisition unit 213 reads the value of each parameter from the robot control parameter 223. Then, the drive control unit 212 performs drive control using the robot control parameters. The parameter acquisition unit 213 acquires the robot control parameter 223 according to the service executed by the mobile robot 20. Specifically, the parameter acquisition unit 213 changes the robot control parameter 223 according to the service information. In this way, the robot control parameter 223 is operated according to the service executed by the mobile robot 20. Accordingly, the mobile robot 20 can appropriately execute the service.

In the robot control parameter 223, a parameter value is set for each service to be executed. That is, the parameter values of the robot control parameters 223 differ depending on the type of service. For example, the storage unit 22 stores a table of the robot control parameters 223 corresponding to the service. Of course, some of the plurality of robot control parameters 223 may be constant parameter values regardless of the type of service.

The service information acquisition unit 216 acquires service information 226 related to a service executed by the mobile robot 20. The parameter acquisition unit 213 acquires the robot control parameter 223 in accordance with the service information 226. That is, the parameter acquisition unit 213 acquires an appropriate parameter value according to the content of the service currently performed by the mobile robot 20 or the next service. That is, the parameter acquisition unit 213 can read out an optimum robot control parameter for each service. Therefore, the mobile robot 20 can provide the service in an appropriate operation.

The service information acquisition unit 216 acquires a plurality of robot control parameters 223 set for each service based on the service information 226. The arithmetic processing unit 21 changes at least one of a virtual bumper distance, a speed, a suspension hardness, a turning radius, an allowable inclination angle, a travelable environment designation, a battery consumption mode, and a wireless sensitivity according to the service information 226. Of course, in two or more services, some of the robot control parameters 223 may have the same parameter value. The drive control unit 212 controls the operation of the mobile robot 20 using the robot control parameters corresponding to the service information 226.

### Example of Setting Robot Control Parameters According to Service Information

Depending on the service, a distance threshold (also referred to as a virtual bumper distance) with respect to a surrounding object is variable. The drive control unit 212 refers to the robot control parameter 223 and detects that the distance indicated by the distance information obtained from the distance sensor group 24 is less than the virtual bumper distance. When the distance indicated by the distance sensor is less than the virtual plate-paper distance, the drive control unit 212 stops or decelerates the operation.

For example, the outer shape changes according to the accessory unit 30 attached to the mobile robot 20. Therefore, the parameter acquisition unit 213 can set different virtual bumper distances according to the accessory unit 30 attached to the mobile robot 20. For example, it is assumed that the size of the transport unit 31 is larger than the size of the cleaning unit 32, the security unit 33, and the guide unit 34. In this case, the arithmetic processing unit 21 sets the virtual bumper distance when the transport unit 31 is mounted to be larger than the virtual bumper distance when the cleaning unit 32, the security unit 33, and the guide unit 34 are mounted. The virtual bumper distance when the mobile robot 20 is executing the transport service is larger than the virtual bumper distance when the mobile robot is executing the cleaning service, the security service, and the guidance service. That is, the parameter acquisition unit 213 updates the virtual bumper distance based on the service information 226.

In addition, when the mobile robot 20 travels in an aisle or the like of a facility, the mobile robot travels straight along the aisle in a state of being separated from the left and right wall surfaces by a predetermined distance. At this time, the robot control parameter 223 may be set so as to change the separation distance from the wall surface according to the size of the accessory unit 30. In this way, when the mobile robot 20 is mounted with the accessory unit 30 having a small size, it is possible to travel through a narrower passage or the like. In a case where the passage width that can be passed varies according to the service information 226, the route planning unit 215 may perform route planning according to the passage width. That is, the route planning unit 215 may perform route planning so as to avoid a passage that cannot pass through.

Alternatively, the turning radius may be variable based on the service information 226. The arithmetic processing unit 21 changes the turning radius of the mobile robot 20 according to the size of the accessory unit 30. As a result, the mobile robot 20 on which the accessory unit 30 is mounted can be appropriately pivoted. In this way, the mobile robot 20 mounted with the accessory unit 30 can stably travel. In a facility where the mobile robot 20 boards the elevator, the number of mobile robots 20 that can board the elevator at the same time may be variable in accordance with the service information 226. That is, in a case where the accessory unit 30 having a large outer shape is mounted, the number of robots that can ride on one elevator is reduced.

Alternatively, the speed information regarding the moving speed may be variable in accordance with the service information 226. Here, the drive control unit 212 controls the drive unit 26 so as to travel at a speed set by the speed information. The speed information may include information such as a speed upper limit value, a speed lower limit value, a speed range, or an acceleration. For example, when the speed upper limit value is set, the drive control unit 212 limits the rotational speed of the drive wheels so that the mobile robot 20 does not move at a speed equal to or higher than the speed upper limit value.

When the accessory unit 30 having a large weight is mounted, the robot control parameter 223 is changed so that the speed upper limit value becomes low. Alternatively, in the case of the security service, the robot control parameter 223 that moves at high speed is set. Accordingly, the mobile robot 20 can reliably detect an intruder. In the case of the cleaning service, the robot control parameter 223 is set so as to move at a low speed. As a result, the mobile robot 20 can reliably suck dust and the like, so that the cleaning service can be appropriately executed.

Alternatively, in the case of the guidance service, the robot control parameter 223 is set so as to have a speed range corresponding to the walking speed. In this case, speed information regarding the lower speed limit value and the upper speed limit value is set as the robot control parameter 223. As a result, the mobile robot 20 can appropriately guide the facility.

In the case of the transportation service, the speed may be changed according to the transported object. In the case of a heavy conveyed object or a fragile conveyed object, the robot control parameter 223 is set so that the speed upper limit value becomes low. Alternatively, when transporting a specimen or fragile medical device, the upper speed limit is set to be low when serving a meal. As a result, the mobile robot 20 can stably convey the conveyed object. On the other hand, in the case of a conveyed object that is light in weight and difficult to break, the robot control parameter 223 is set so that the speed upper limit value becomes high. Alternatively, in a case where the used tableware is to be placed on the lower table, the robot control parameter 223 is set so that the upper limit value of the speed becomes high. As a result, the mobile robot 20 can efficiently convey the conveyed object. In this case, the service information 226 may include information for identifying the conveyed object.

The range of the allowable inclination angle may be variable in accordance with the service information 226. The allowable angle range is, for example, an allowable range of the roll angle or the pitch angle of the chassis. For example, an angle sensor or an angular velocity sensor mounted on the chassis detects an angle of the chassis. When the allowable inclination angle range is exceeded, the mobile robot 20 stops or decelerates. When the mobile robot 20 is mounted with a wagon that is the transport unit 31, the center of gravity increases. On the other hand, when the mobile robot 20 is equipped with a vacuum suction cleaner that is the cleaning unit 32, the center of gravity is lowered. Therefore, when the conveyance service is executed, the allowable inclination angle range may be set to be smaller than when the cleaning service is executed. By doing so, it is possible to prevent the mobile robot 20 from traveling in an unstable state.

In addition, the battery consumption mode may be variable in accordance with the service information 226. When the accessory unit 30 consumes a large amount of power, the power consumption of the drive motor of the wheel is reduced as the power saving mode. As a result, it is possible to suppress a sudden power consumption.

In addition, the sensitivity of the wireless signal may be variable in accordance with the service information 226. For example, the accessory unit 30 to be mounted may hinder transmission and reception of the wireless signal depending on the size and shape thereof. Therefore, the reception sensitivity, the transmission intensity, and the transmission direction of the wireless signal may be changed in accordance with the service information. For example, it is assumed that the mobile robot 20 has a plurality of radio signal transceivers, and the frequency bands thereof are different from each other. For example, it is assumed that the mobile robot 20 includes a wireless communication device in 2.4 GHz band and a wireless communication device in 5 GHz band. The mobile robot 20 may switch the wireless communication device to be used according to the service information 226. In this way, the mobile robot 20 can change the sensitivity of the wireless signal according to the service information 226.

At least one robot control parameter is changed in response to the service information 226. Of course, more than one robot control parameter may be changed. In this way, each service can be appropriately executed by the mobile robot 20. For example, when the mobile robot 20 autonomously travels, the mobile robot 20 is autonomously controlled by the robot control parameter 223 suitable for the service. In this way, the mobile robot 20 can autonomously travel appropriately. Even when the mobile robot 20 is used together with the accessory unit 30, the management system 1 can perform appropriate control according to the service. Of course, at least a part of the processing of the mobile robot 20 may be performed by the host management device 10.

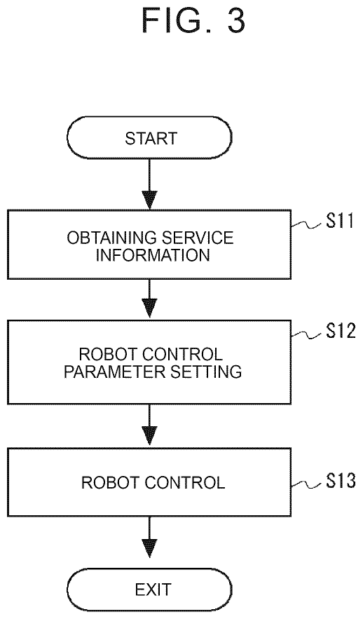

FIG. 3 is a flowchart illustrating a management method. The management method manages the mobile robot 20 and a plurality of accessory units that are used in combination with the mobile robot 20 to enable the mobile robot to execute a plurality of different services.

The service information acquisition unit 216 acquires service information related to a service executed by the mobile robot 20 (S11). The parameter acquisition unit 213 sets a plurality of robot control parameters set for each service based on the service information (S12). The arithmetic processing unit 21 controls the operation of the mobile robot 20 in accordance with a plurality of robot control parameters (S13). In this way, even when the mobile robot 20 is used together with the accessory unit 30, the management system 1 can perform appropriate control according to the service. Of course, at least a part of the processing of the mobile robot 20 may be performed by the host management device 10.

Further, the robot control parameters 223 may be adjustable by a user or an administrator. For example, when the user or the like selects the adjustment of the robot control parameter 223, the user terminal 400 displays a bar or the like for the adjustment. The control parameters are updated by the user or the like operating the user terminal 400. The robot control parameters 223 may be updated for each service. When the parameter acquisition unit 213 acquires the updated value, the value of the robot control parameter 223 in the storage unit 22 is rewritten. Then, the arithmetic processing unit 21 controls the operation of the mobile robot by using the updated robot control parameters. In this way, the mobile robot 20 controls the operation using more appropriate robot control parameters 223. When the mobile robot executes the same service again, the mobile robot 20 performs control using the updated robot control parameter 223.

The mobile robot 20 may use a machine learning model such as deep learning in the control of the route planning and the drive control unit 212. Further, in detection of a surrounding object or the like, a machine learning model such as deep learning such as RNN (Recurrent Neural Network) or CNN (Convolutional Neural Network) may be used.

In addition, some or all of the processing in the mobile robot 20, the host management device 10, and the like described above can be realized as a computer program. The program as described above is stored using various types of non-transitory computer-readable media, and can be supplied to a computer. The non-transitory computer-readable media include various types of tangible recording media. Examples of the non-transitory computer-readable media include magnetic recording media (e.g. flexible disks, magnetic tapes, hard disk drives), magneto-optical recording media (e.g. magneto-optical disks), compact disc read-only memory (CD-ROM), compact disc recordable (CD-R), compact disc rewritable (CD-R/W), and semiconductor memory (e.g. mask ROM, programmable ROM (PROM), erasable PROM (EPROM), flash ROM, random access memory (RAM)). The memory is an example of a storage medium. Further, the program may also be supplied to the computer by various types of transitory computer-readable media. Examples of the transitory computer-readable media include electrical signals, optical signals, and electromagnetic waves. The transitory computer-readable media can supply the program to the computer via a wired communication path such as an electric wire and an optical fiber, or a wireless communication path.

The present disclosure is not limited to the above embodiment, and can be appropriately modified without departing from the spirit.

What is claimed is:

1. A management system that manages an autonomous mobile robot and a plurality of accessory units to be used in combination with the autonomous mobile robot to enable the autonomous mobile robot to execute a plurality of different services, the management system being configured to:
acquire service information related to a service executed by the autonomous mobile robot;
acquire a plurality of control parameters set for each service based on the service information; and
control operation of the autonomous mobile robot according to the control parameters.

2. The management system according to claim 1, wherein the control parameters include at least one of a speed, a height, a virtual bumper distance, a suspension hardness, a turning radius, an allowable inclination angle, a travelable environment designation, a battery consumption mode, and a wireless sensitivity.

3. The management system according to claim 1, wherein the accessory units include at least one of a transport unit, a cleaning unit, a security unit, and a guide unit.

4. A management method of managing an autonomous mobile robot and a plurality of accessory units to be used in combination with the autonomous mobile robot to enable the autonomous mobile robot to execute a plurality of different services, the management method comprising:
acquiring service information related to a service executed by the autonomous mobile robot;
acquiring a plurality of control parameters set for each service based on the service information; and
controlling operation of the autonomous mobile robot according to the control parameters.

5. A non-transitory storage medium storing a program that causes a computer to execute a management method of managing an autonomous mobile robot and a plurality of accessory units to be used in combination with the autonomous mobile robot to enable the autonomous mobile robot to execute a plurality of different services, wherein the management method comprises:
acquiring service information related to a service executed by the autonomous mobile robot;
acquiring a plurality of control parameters set for each service based on the service information; and
controlling operation of the autonomous mobile robot according to the control parameters.
